(19) United States
(12) Patent Application Publication
(10) Pub. No.: US 2026/0074078 A1
(43) Pub. Date: Mar. 12, 2026

(54) COMPUTER SYSTEM AND METHOD FOR PROVIDING A SUBJECT-RELATED DATA DEVELOPMENT PLATFORM

(71) Applicant: LizAI Inc., Newton, MA (US)

(72) Inventors: David R. Elmaleh, Newton, MA (US); Duy Khiet Ho, Quincy, MA (US); Trung Tin Nguyen, Saarbrücken (DE)

(73) Assignee: LizAI Inc., Newton, MA (US)

(21) Appl. No.: 19/087,980

(22) Filed: Mar. 24, 2025

Related U.S. Application Data
(63) Continuation-in-part of application No. 18/828,300, filed on Sep. 9, 2024.

(51) Int. Cl. G16H 50/70

(52) U.S. Cl. CPC G16H 50/70

(57) ABSTRACT

A method comprises receiving at least one input data object containing subject-related information according to at least one of information types encoded in at least one of data formats; and processing the at least one input data object for standardizing the subject-related information. The method further includes subjecting the subject-related information to a first machine learning model for generating a uniform dataset containing the subject-related information in a uniform structured format; storing the uniform dataset in one or more secured data repositories connected to a network; and providing a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.

### CROSS-REFERENCE TO RELATED APPLICATIONS

The present application is a continuation-in-part of and claims the benefit of priority to U.S. patent application Ser. No. 18/828,300, filed Sep. 9, 2024, which is incorporated herein by reference in its entirety for all purposes.

### BACKGROUND OF THE DISCLOSURE

In subject-related development fields, in particular the life sciences, such as biotechnology, medicine and healthcare, though not limited thereto, availability of systematic information, including factors and causalities, is vital, for example, for the development of targeted and/or individualized processes, such as medical treatment schemes, and/or agents, such as drugs. However, obtaining systematic information is often difficult in such fields due to the complexity of the processes involved and diverse individual conditions in real scenarios. Systematic knowledge is often achieved in such fields by combining large amounts of data, which, in turn, is often not available to either enterprise, corporation, or private users, including private individuals, commercial developers of subject-related processes and/or products, etc.

Furthermore, while a large amount of subject-related data mostly exists, for example, sample measurement records, individual reports and diagnostics, individual prescriptions, large-scale (demoscopic) surveys, public statistics, published research articles, etc., such data varies regarding the information type which it represents, including audio information, photographic information, handwritten notes, printed text, electronic character strings, etc., and regarding its accessibility, for example, maintained in an individual's private domain, published on the Internet, etc.

Furthermore, especially in a health-related context, but also in other subject-related fields, increased privacy regulations typically apply concerning a use of personal information. In addition, many individuals prefer not providing their personal subject-related data to third parties. This poses further burdens to subject-related data development.

### BRIEF SUMMARY OF THE DISCLOSURE

There is thus a need for a technique that facilitates improved subject-related data development.

Accordingly, there is provided a method according to claim 1, a computer program product according to claim 15, and a computer system according to claim 16.

According to an aspect, a method, performed by a computer system connected to a network, is provided. The method comprises receiving, by means of a data receiving module of the computer system and from at least one of a plurality of sources connected to the network, at least one input data object containing subject-related information according to at least one of a plurality of information types encoded in at least one of a plurality of data formats. The method further comprises processing, by means of a data extraction and classification module of the computer system, the at least one input data object for standardizing the subject-related information; subjecting, by means of a data engineering module of the computer system, the subject-related information contained in the processed at least one input data object to a first machine learning model for generating a uniform dataset containing the subject-related information in a uniform structured format; and, storing, by means of a storing module of the computer system, the uniform dataset in one or more secured data repositories connected to the network. The method further comprises providing, by means of a workspace module of the computer system, a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.

Thus, the method permits provision of a data development platform, or workspace, which, by means of a correspondingly configured data extraction and classification module, is subject-related, and which is accessible for users, for example, by means of user communication devices, via a communication network, such as the Internet. As such, the method facilitates accessibility of the subject-related data development platform to a large group of potential users. An applicability of the method can thus be extended.

Furthermore, implementation of the method in connection with a networked environment facilitates receiving input data objects from a wide range of network-connectible sources for establishing the data development platform, for example, for training one or more machine learning models which may be used in performing one or more steps of the method. In addition, a processing of data objects containing subject-related information in any of multiple information types, such as acoustic information, image information and/or text information, can be facilitated by means of a correspondingly configured data extraction and classification module, thus further increasing a range and an amount of the subject-related information available. An effectiveness of the method, in particular a reliability and a suitability of data which is generated by means of the secured virtual environment, can be improved.

Furthermore, implementation of the method by means of a computer system facilitates an implementation, by an operator of the computer system, of (such as general) security rules, including privacy rules, regarding information contained in any data object which is received, stored and/or generated using the method and/or regarding an authorization of an individual user to access some or all of the functionalities of the method. This applies, in particular, in connection with data objects which are stored in the domain of an operator of the computer system.

Additionally, or alternatively, the method facilitates that any user connected to the computer system via the network is enabled to select individual security or privacy settings, for example, via a user terminal associated with a respective user, regarding subject-related data objects which are transmitted from the user, for example, by the user's own user terminal, to the computer system and/or which are generated using data objects relating to the user. In this way, a user is enabled to control a degree of privacy regarding information contained in data objects which may be transmitted to the computer system, thus increasing an acceptance of the method by a larger group of users and/or compliance of the method with legal security regulations.

Furthermore, the method facilitates that at least some functionalities of the method, in particular some or all of the processing, subjecting, storing and/or providing steps, are performed in a domain of a user, for example, on a user terminal associated with a respective user. In this way, privacy of personal information contained in data objects associated with the user can be further improved, since personal or user-specific data may not need to be transmitted via the network. To this end, a provider of the method may provide corresponding computer software for installation on the user terminal(s). In such implementations, the user terminal(s) may be regarded as an extension of the computer system, which thus extends at least partially in the domain of the user(s).

Furthermore, by standardizing the subject-related information and generating, by means of a machine learning model, a uniform dataset containing the subject-related information in a uniform structured format, such as a tabular format organized by predefined subject-related information classes, the method facilitates a use of each uniform dataset, and of the information encoded therein, for any of various applications in connection with the one or more user-controlled subject-related data development operations for generating at least one workspace-developed data object. This may apply, in particular, independently from a specific information content of the received data object and of the information type. An application range, an effectiveness, and a usability of the method for subject-related data development can thus be further improved.

The method may further comprise: Processing, by means of multiple data extraction and data structure of multiple data inputs.

In an embodiment, extracting information, including structured data, unstructured data, and/or semi-structured data (for example pdf, DICOM, notes, image, and/or test results) from tables for all subjects in an institution or government and/or to produce sets of data per disease within a subject or with a group of subjects may be performed by the method. These sets may allow learning, analyzing, and/or predicting the health of individuals, groups (hospitals), institutions and/or government to predict people's health. For example, the information may comprise multiple unstructured data. Alternatively, or in addition, the information may comprise multiple structured data, unstructured data, and/or semi-structured data.

In an embodiment, means for learning about the data (such as the structured and unstructured data) within subject and within subjects are provided within the method. For example, the means for learning about the data may be applied on one or more data. In addition, an information may be obtained which then may be evaluated and/or used in the method. This may have a beneficial impact on insights and/or may allow to sync of the information of all subjects.

In an embodiment, particularly in a health-related context, but also other subject-related fields, means for mega-collection and mega-structure of meaningful data from fragmented sources across levels from hospitals to health management organizations, such as department of health, and ministry of health, are provided. This is highly beneficial in order to develop insights in the data. The mega-structure of health data may comprise technologies which can ingest, process, retrieve, and/or structure meaningful data and then optionally organize in formats, such as table, knowledge graph, and/or knowledge interaction. These technologies may include, for example, large language models (LLMs) with advanced retrieval-augmented generation (RAG) techniques, self-supervised AI models, Vision Transformers (ViTs), Optical Character Recognition (OCR) enhanced by Vision Transformers (ViTs), multi-modal deep learning, Graph Neural Networks (GNNs), heterogeneous biomedical knowledge graphs, federated learning with differential privacy, and/or distributed data pipelines with secure multi-party computation. For example, the mega-structure of health data may start from department, hospital levels to enterprise, national, and even international levels. Thus, individuals may benefit from precision diagnosis, prognosis, and treatment, as doctors have insights into all data, while the higher level managers, such as institutions, and government, may improve their management efficiency as well as predict disease progression and resolve its effect on society, like epidemic or pandemic events.

In an embodiment, the method may comprise: Mega-structuring data, such as the subject-related information contained in the at least one input data object, from multiple sources, especially of any kinds, and/or from any management levels, such as clinics, hospitals, ministry of health, and/or countries, into a table form. An exemplary result data provided (especially by the methods and systems described herein) may provide for one or more of the following information and/or functionalities 1.-6. (for example, at least in part in form of a spreadsheet table).
1. A column, such as a first column, is an anonymized subjects'number from 1 to N (with for example N=361,742,591, which is the US population, just as an example);
2. A horizontal row imports all defined diseases (for example from 1 to M, with for example M=10,000, which is the total recorded diseases, just as an example), indicating how many diseases a person suffers from and/or how the disease spreads within the population. A summary for each disease may be provided at the bottom of all subjects;
3. Pressing on a disease X (cancer, for example) opens a spread that indicates data and/or health-related data (especially all health-related data), for example symptoms, blood tests, MRIs, PET, drug treatments, imaging, PDFs, and/or other tests. A column, such as the first column, may be the anonymous subject number of the tests and the rows;
4. Pressing on an information (MRI image, for example) opens the history of all information related to that information (further MRI images, for example) for each subject; the same may be provided for other tests. Pressing the drug treatment may, alternatively or in addition, presents all the concomitant drugs the subject takes;
5. In a separate table, diagnosis, prognosis, drug side effects, morbidity, etc. may be provided; and/or
6. Suitability of the result data, such as a structured table of health-related data, to be used in large-scale medical research, and/or to be used during training of AI model, especially for improving predictive analytics by enhancing AI model training.

Using the result data, such as a structured table of health-related data, may facilitate large-scale medical research, and/or may improve predictive analytics by enhancing AI model training.

Providing such information, the present disclosure allows to provide detailed analysis means. Of course, the example described above could be modified to new or improved data control. It is preferred that the users utilize their internally dedicated servers. This way the security may be in the hands of the users.

The plurality of information types may comprise at least one of image information, textual information, acoustic information, voice information, spreadsheet data, and/or database information, the database information including at least one of data generated by software and/or hardware, relational database information, object-oriented database information, and/or NoSQL database information.

The uniform dataset may be generated such that the uniform dataset complies with at least one privacy and/or security standard defined by legal regulations in one or more jurisdictions regarding the subject-related information.

The subject-related information contained in the received at least one input data object may be subjected to a personal data anonymization module, for performing anonymization of personal data contained in the subject-related information. The subjecting and performing anonymization may be made, for example, prior to processing, by means of the data extraction and classification module of the computer system, the at least one input data object.

Standardizing the subject-related information may comprise applying an error-detection-and-correction routine to the subject-related information.

The plurality of sources may comprise a plurality of source types.

The uniform structured format may comprise a uniform category-mapped format, in particular a uniform category-mapped tabular, diagram, chart and/or figure format.

The data development operations may include subjecting the imported datasets to at least one second machine learning model comprised by the workspace module.

The subject-related information may be life sciences-related, in particular health-related, information. Additionally, or alternatively, the subject-related data development operations may be life sciences-related, in particular health-related, data development operations.

Additionally, or alternatively, the subject-related information may be information from multiple domains, in particular comprising life sciences-related, more particularly health-related, health maintenance organizations-related, pharmaceutical technologies-related, biology-related and/or biotechnologies-related information. Additionally, or alternatively, the subject-related data development operations may be for data from multiple domains and comprise life sciences-related, in particular health-related, health maintenance organizations-related, pharmaceutical technologies-related, biology-related and/or biotechnologies-related data development operations.

The user-controlled subject-related data development operations may comprise receiving, from a user via the network, a life sciences-related, in particular health-related, pharmaceutical technologies-related, biology-related, biotechnologies-related, query, and the at least one workspace-developed data object comprises a life sciences-related, in particular health-related, pharmaceutical technologies-related, biology-related, biotechnologies-related, response to the life sciences-related query, the life sciences-related response for output by the computer system.

The life sciences-related query may be a health-related query, and the life sciences-related response may be a health-related response. Additionally, the health-related query and the health-related response may relate to at least one of a clinical condition and information of a person, medical information, a description of a medication, an interaction of medications, a mechanism of action of a medication, an underlying cause of a disease, a prediction of a disease development, a prevention of a disease development, medical treatment, personalized treatment, best fit treatment, a biological target for a treatment, and/or drug development.

The plurality of information types may comprise at least one of a handwritten note by a medical professional, a medical image, an electronic healthcare record, medical spreadsheet data, and/or medical database information, the medical database information including at least one of clinical data generated by software and/or hardware, relational database information, object-oriented database information, and/or NoSQL database information.

The life sciences-related information may be a health-related data. The health-related data may comprise multiple structured, and/or unstructured and/or semi-structured formats. The health-related data may comprise individual personal data which may or can be stored at hospital or clinic departments, clinics, hospitals, institutions and/or at least one of the health maintenance organizations managed by the governments. The individual personal data may be collected from regions, states, countries, and/or the globe.

The one or more secured data repositories may comprise one or more secured data repositories hosted by an operator of the computer system and/or one or more secured data repositories hosted at one or more secured user domains of one or more users of the computer system.

According to another aspect, a computer program product is provided. The computer program product contains portions of program code which, when executed by a processor of a computer system, configure the computer system to perform the method as provided herein.

According to another aspect, a computer system is provided. The computer system comprises a processor and a data storage device operatively coupled to the processor, the data storage device containing portions of program code which, when executed by the processor, configure the processor to perform the following steps: receiving, by means of a data receiving module of the computer system and from at least one of a plurality of sources connected to a network, at least one input data object containing subject-related information according to at least one of a plurality of information types encoded in at least one of a plurality of data formats; processing, by means of a data extraction and classification module of the computer system, the at least one input data object for standardizing the subject-related information; subjecting, by means of a data engineering module of the computer system, the subject-related information contained in the processed at least one input data object to a first machine learning model for generating a uniform dataset containing the subject-related information in a uniform structured format; storing, by means of a storing module of the computer system, the uniform dataset in one or more secured data repositories connected to the network; and, providing, by means of a workspace module of the computer system, a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Further details and advantages of the disclosure become apparent from the detailed description and the drawings. There is shown in:

FIG. 1 a computer system according to an example,

FIG. 2 a method according to an example,

FIG. 3 processing an input data object by means of a data extraction and classification module according to an example,

FIG. 4 an illustration of a data mega-structure according to an example,

FIG. 5 an illustration of a workflow of a method according to an example,

FIG. 6 an illustration of a data mega-structure according to another example,

FIG. 7 an illustration of a table of a disease,

FIG. 8 an illustration of a selection of imaging for primary staging, and

FIG. 9 an illustration of a fully secured AI-powered platform according to an example.

### DETAILED DESCRIPTION OF THE DISCLOSURE

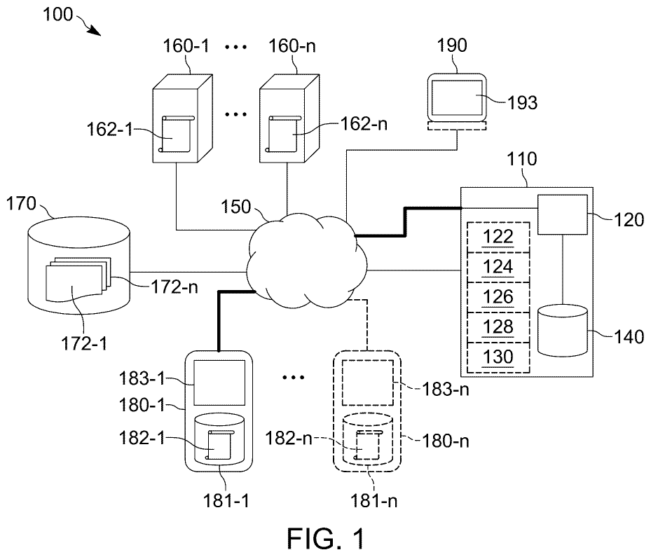

FIG. 1 shows schematically and exemplarily a networked computing environment 100. The networked computing environment 100 comprises a computer system 110, which is communicatively coupled to a network 150, such as the Internet.

The computer system 100 includes a processor 120 and a data storage device 140 operatively connected to the processor 120. The data storage device 140 stores program code which is executable by means of the processor 120. When the processor 120 executes the program code, the processor 120 performs operations which functionally constitute a data receiving module 122, a data extraction and classification module 124, a data engineering module 126, a storing module 128, and a workspace module 130, as will be described in more detail below.

Further connected to the network 150 is a plurality of network nodes, which include multiple servers 160-1, 160-2, 160-n. Each of the servers stores at least one data object 162-1, 162-2, 162-n, which pertain to one or more data repositories hosted by the servers 160-1, 160-2, 160-n. In some examples, at least some of the data objects 162-1, 162-2, 161-n pertain to a same data repository, such as a database which is managed by a third party and which is hosted in a distributed manner by various of the servers 160-1, 160-2, 160-n. Moreover, in some examples, each of at least some of the data objects 162-1, 162-2, 162-n belongs to a different data repository. The data objects 162-1, 162-2, 162-n are accessible via the network 150, for example, by means of a network browser of a computer system connected to the network 150, such as the computer system 110. In some examples, the data objects 162-1, 162-2, 162-n include one or more websites, which can be accessed and rendered by means of a network browser, such as public Internet websites, Internet websites with privileged access, etc.

The networked computing environment 100 further includes a user terminal 180-1 which is communicatively connected to the network 150. The user terminal 180-1 may include any type of user device which is suited for communicating data via the network 150, outputting received data towards a user via a user interface 183-1 of the user terminal 180-1 and receiving user input data from the user via the user interface 183-1 and transmitting received user input data via the network 150. Those types include mobile computing devices, such as smartphones, notebooks and/or tablet computers, stationary user terminals, such as computer stations with a user interface, etc.

The user terminal 180-1 comprises a data storage device 181-1, such as a built-in computer-readable non-volatile memory device. In the shown example, at least one subject-related data object 182-1 is stored on the data storage device 181-1. The user terminal 180-1 is configured to transmit the data object 182-1 towards components of the computer system 110 for use in a method as described herein. In some examples, the subject-related data object 182-1 contains encoded therein subject-related information which comprises personal and/or user-specific information of a user associated with the user terminal 180-1.

As indicated in FIG. 1 by the double lines, a data connection between the user terminal 180-1 and components of the computer system 110 via the network 150 is in some examples implemented as a secured data connection. In this way, security of transmitted user-related data and of data objects 182-2 stored by the user terminal 180-1 from unauthorized accessing is improved.

In an example, the subject-related information encoded in the comprises health-related information of a user of the user terminal 180-1, such as one or more of a medical record, diagnostic image data, a digitalized hand-written note by a doctor, a medical prescription, etc.

As indicated by the dashed lines in FIG. 1, the networked computing environment 100 may in some examples optionally include additional user terminals 180-n. Each of the additional user terminals 180-n is communicatively connected to the network 150 and comprises a data storage device 181-n with one or more subject-related data objects 182-n is stored thereon.

As indicated by the dashed lines in FIG. 1, the networked computing environment 100 may in some examples optionally include at least one subject-related, non-public database 170. The non-public database 170 stores non-public information which is encoded in one or more non-public data objects 172-1, 172-n. In an example, the non-public database 170 is a clinical database storing information about one or more patients, such as patient-specific bio-parametric data, patient-specific health data, patient-specific medical treatment data, non-patient-specific health data, non-patient-specific medical treatment data, etc., which are encoded in non-public data objects 172-1, 172-n.

The networked computing environment 100 further includes a developer terminal 190 which is communicatively connected to the network 150. The developer terminal provides in some examples the same functionalities as the user terminal 180-1 and may include any type of device which is suited for communicating data via the network 150, outputting received data towards a developer user via an interface 193 of the developer terminal 190 and receiving developer input data from the developer user via the interface 193 and transmitting received developer input data via the network 150.

In some examples, the developer terminal 190 is associated with a developer use who uses some or all of the functionalities provided by the computer system 110, as described below, without providing to the computer system 110 any subject-related data objects for processing in accordance with the techniques described herein. The developer user is, for example, a process and/or product developer, who uses the capacities of a virtual workspace provided by the computer system 110, including processed, in some examples anonymized, subject-related data which is stored by the computer system 110. The developer user is, for example, an authorized, such as registered, user of the functionalities offered by the computer system 170.

When in operation, the computer system 110 is configured to receive, by means of the data receiving module 122, input data objects containing subject-related information. The data objects include data objects such as the data objects 162-1-162-n stored by means of servers 160-1-160-n, data objects 172-1-172-n stored by means of nonpublic database 170, and/or data objects 182-1-182-n stored by means of user terminals 180-1-180-n. The subject related information comprises information relating to a subject for which the computer system has been configured to perform the techniques described herein, for example, life science-related information, health-related information, or the like.

In examples in which the subject-related information comprises health-related information, the data objects 162-1-162-n comprise one or more of published health statistics, published medical research, published medical surveys, and the like. Additionally, or alternatively, the data objects 172-1-172-n comprise nonpublic institutional medical data, such as clinical data. Additionally, or alternatively, the data objects 182-1-182-n comprise personal health-related data pertaining to a user associated with a respective user terminal 180-1-180-n, such as personal diagnostic data, digitalized handwritten notes by doctors, individual medical prescriptions, individual medical records, and the like.

In some examples, the data receiving module 122 is configured to receive data objects which have been transmitted from any of the network nodes, for example, upon input by a user of the corresponding network node, towards the data receiving module 122. Additionally, or alternatively, in some examples, the data receiving module 122 is configured to actively retrieve data objects, for example, by browsing data depositories connected to the network 150.

The computer system 110 is further configured to process each received input data object by means of the data extraction and classification module 124. The data extraction and classification module 124 is configured to process a range of coding formats and information types represented by the data objects, such as image information, handwritten notes, digital text, acoustic information, and the like, and to extract subject-related information encoded therein. To this end, the data extraction and classification module 124 employs in some examples optical character recognition as well as one or more machine learning models which have been trained for this purpose, etc.

In some examples, the data extraction and classification module 124 is further configured to identify subject-related entities in the processed data objects, such as, drugs, diseases, side effects, when used in the health-related context, and to generate metadata for each data object in which one or more of the identified entities is indicated.

In some examples, the data extraction and classification module 124 is further configured to classify the data object being processed based on the information types, identified entities, user characteristics, and the like, in accordance with any of multiple relevant categories of information for the subject-related data development platform, such as medical scans, doctor notes, or disease types, when used in a health-related context.

In some examples, the data extraction and classification module 124 is further configured to standardize the subject related information, including performing an error-detection-and-correction routine on the subject related information, and to organize and enrich the extracted and classified data for efficient queuing and analyzing, in particular to create and manage a structured representation of the processed information, for example, as part of a knowledge graph building process.

The computer system 110 is further configured to subject the subject-related information in the processed input data object to a machine learning model for generating a uniform data set which contains the subject-related information in a uniform structured format. To this end, the computer system 110 comprises a correspondingly configured data engineering module 126. Subjecting the information of the processed input data object to a generating of a uniform data set facilitates future use of the information, for example, in automatically creating a response to a user query.

In an example, the uniform structured format corresponds to a tabular format, in which the subject-related information is included, in the manner of an information template, in accordance with information classes. For example, when used in a health-related context, the information classes include different treatment parameters in a history of medical treatment of an individual user, or the like.

The computer system 110 is further configured to store, by means of the storing module 128, the uniform data set in a secure data repository 140, 181-1-181-n. In the case of personalized data, in some examples, the storing module 128 is configured to store the uniform data set in a secure storage device within a domain of the respective user, such as on data storage device 181-1 of the user terminal 180-1. In the case of anonymized data, which may be intended to be available to a process developer or a drug developer, such as developer user of the developer terminal 190, in some examples, the storing module 128 is configured to store the uniform data set in a secure storage device in a domain of an operator of the computer system 110, such as the data storage device 140.

The computer system 110 is further configured to provide, by means of the workspace module 130, a secure virtual environment accessible to users, such as users of the developer terminal 190 and/or users of any of the user terminals 180-1-180-n. The secure virtual environment, as exemplarily represented in FIG. 1 by the interfaces 183-1-183-n, 193, enables the importation of data sets stored in the secured data repositories 140, 181-1-181-n and the use of the imported data sets as part of user-controlled subject-related data development operations. Accessibility of a stored dataset to a particular user is determined, in some examples, depending on an authorization of the specific user in relation to a specific dataset and/or accessibility of the stored data set at its location of storage from the perspective of a given user device.

The user-controlled subject-related data development operations are configured for generating workspace developed data objects. For example, when used in a health-related context, the user-controlled subject-related data development operations include a personal user query which is input by a user of any of the user terminals 180-1-180-n, which results in a health-related response being generated and provided by means of the secure virtual environment. In another example, the user-controlled subject-related data development operations include requirements specifications being input by a drug developer, which results in product specifications generated by means of the secure virtual environment, and the like.

In some examples, the workspace module 130 includes at least one machine learning model for performing and/or supporting the data development operations executed on the imported data sets.

The above examples have been described in connection with FIG. 1, in which the computer system 110 is shown as a separate network entity. However, it will be understood that in other examples some or all of the above-described functionalities of the computer system 110 can be implemented on one or more of the user terminals 180-1-180-n and/or the developer terminal 190, respectively. For example, a provider of the method described herein provides software code to authorized users of the user terminals 180-1-180-n and/or of the developer terminal 190, wherein such software code is executable by means of the user terminals 180-1-180-n and/or the developer terminal 190 to configure the user terminals 180-1-180-n and/or the developer terminal 190 to perform some or all of the operations described herein.

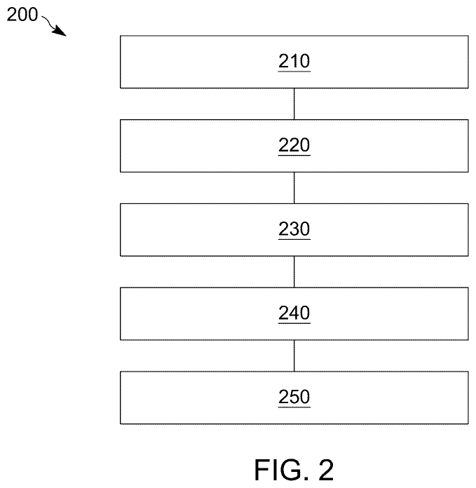

FIG. 2 shows a flow diagram of a method 200 performed by computer system connected to a network, such as computer system 110 in the networked computing environment 100 as described above in connection with FIG. 1.

The method 200 includes receiving, by the computer system and from at least one of a plurality of sources connected to the network, at least one input data object containing subject-related information according to at least one of a plurality of information types encoded in at least one of a plurality of data formats, step 210.

The method 200 further includes processing, by means of a data extraction and classification module of the computer system, the at least one input data object for standardizing the subject-related information, step 220. An example implementation of step 220 is described in more detail below in connection with the method 300.

The method 200 further includes subjecting, by means of a data engineering module of the computer system, the subject-related information contained in the processed at least one input data object to a first machine learning model for generating a uniform dataset containing the subject-related information in a uniform structured format, step 230, and, storing, by means of a storing module of the computer system, the uniform dataset in one or more secured data repositories connected to the network, step 240.

The method 200 further includes providing, by means of a workspace module of the computer system, a secured virtual environment accessible to users connected to the network, step 250. The secured virtual environment enables importation of datasets stored in the one or more secured data repositories and a use of imported datasets as part of one or more user-controlled subject-related data development operations for generating at least one workspace-developed data object.

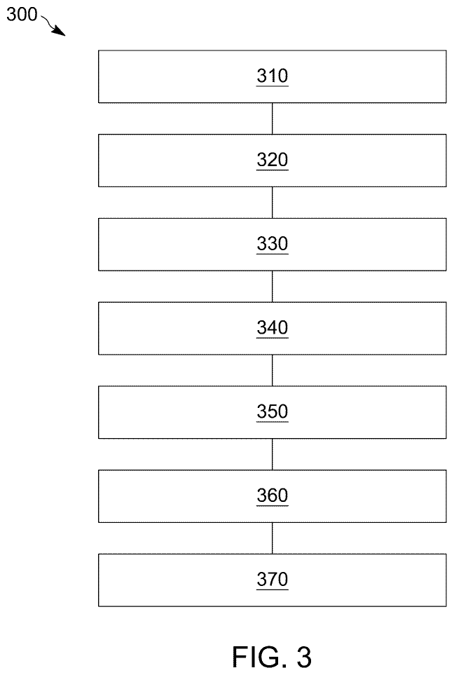

FIG. 3 shows a flow diagram of a method 300 for processing an input data object by means of a data extraction and classification module of a computer system. The method 300 provides an example implementation of step 220 of the method 200.

Upon receipt of the input data object by the data extraction and classification module, such as the data extraction and classification module 124, the method 300 comprises classifying a data format of the input data object, step 310. As a result, an information type of information contained in the input data object can be determined, such as image information, textual information, acoustic information, voice information, spreadsheet data, and/or database information.

The method 300 further comprises performing a structure analysis on the input data object, step 320. For example, when it has been determined in step 310 that the input data object comprises a PDF document, a layout analysis is performed on the PDF document. In another example, when it has been determined in step 310 that the input data object comprises database information, a database schema analysis for the database is performed.

The method 300 further comprises extracting data from one or more detected components of the input data object, step 330. For example, when the input data object comprises a PDF document, text and/or data is extracted from tables, figures, footers, editors, etc., which have been determined as a result of a layout analysis in step 320. In another example, when the input data object comprises database information, data is extracted from tables which have been determined as a result of a database schema analysis in step 320.

The method 300 further comprises detecting data extraction errors and correcting extracted data, using one or more machine learning models, step 340. For example, one or more machine learning models are used to correct errors which result from broken layouts, hard-to-extract handwriting, etc., and/or for improving data accuracy and context. In addition, various other types of errors are corrected in some examples, such as topographical mistakes, overlapping text, font problems, and the like.

The method 300 further comprises combining extracted data from one or more components, step 350. For example, an input data object can have multiple components from which data has been extracted in the preceding steps. Such data is combined in step 350.

The method 300 further comprises identifying domain specific entities and generating metadata, such as annotating or aliasing, for the extracted data, step 360. For this purpose, in some examples, a recognition of named entities and/or clinical variable identification is performed. This is facilitated, in some examples, by provision of a comprehensive list of entities and/or clinical information which can be identified. For example, when the method is used in a medical context, named entities such as drugs, diseases, side effects, relationships among the aforesaid entities, doses, and the like, can be recognized from handwritten doctor notes and, for example, can be converted into a structured format such as JSON.

The method 300 further comprises exporting the extracted data into an electronic file for subsequent operations, step 370. Subsequent operations comprise in some examples the generation of a uniform dataset by subjecting the electronic file to one or more machine learning models, in accordance with step 230 of the method 200.

In a first example scenario, the methods and systems described herein provide, for each user of a first user group, a secure and private virtual environment that allows a user to utilize the operator's built-in machine learning algorithms for visualizing and analyzing subject-related data, developing predictive models, or creating their own machine learning algorithms to work with subject-related uniform datasets. In such a scenario, the user-controlled subject-related data development operations for generating a workspace-developed data object in step 250 comprise, for example, generating a graphical data object which visualizes an analysis of the subject-related data, generating a predictive model or a machine learning algorithm, and the like.

In a second example scenario, the methods and systems described herein provide, for each user of a second user group, a trained machine learning-based assistant which is optimized for subject-related data searching, designed to be effectively securable by security and privacy technologies. It allows a user to efficiently locate and retrieve subject-related information from subject-related input data objects. Using at least one machine learning model for one or more of the processes described ensures that subject-related data remains secure and private while providing accurate insights and easy navigation through large volumes of data. In such a scenario, the user-controlled subject-related data development operations for generating a work-space-developed data object in step 250 comprise, for example, generating a graphical data object which visualizes the located and retrieved subject-related information, and the like. The second example scenario can be implemented simultaneously with the first example scenario, and the first user group and the second user group can overlap at least partially.

In the following, further details of preferred embodiments of the present disclosure (especially the methods and systems described herein) are provided.

The present disclosure may improve diagnostic accuracy, reduce physician burnout, enhance precision treatment, and/or improve management across levels from clinics, insurance companies, to government, in healthcare.

In some embodiments, the subject-related information may be healthcare data composes of any information about a patient's health and medical history, including but not limited to personal information, medical records, clinical data, and/or treatment plans. Clinical data, in turn, may cover lab results, X-rays, diagnostic imaging, and more, of each individual, which exists in various formats, such as .txt, .doc, .xls, .pdf, DICOM, etc.

In some embodiments, the data extraction and classification module may comprise and/or make us of at least one of: natural language processing (NLP), image processing, large language models (LLM), and/or advanced retrieval and data insights.

The present disclosure (especially the methods and systems described herein) may provide for mega-structuring a largely fragmented clinical database into a comprehensive table of all anonymized patients, and variables per disease. The clinical data mega-structure can be designed in any forms beyond table, such as charts, graphs, knowledge interaction, and more, which further provides a maximum accuracy for clinical data semantic search and management across various sources. For example, the data engineering module together with the first machine learning model (especially after processing the at least one input data object by means of the data extraction and classification module) may be used for ultimately obtaining the clinical data mega-structure in the desired forms so as to obtain the uniform dataset containing the subject-related information in a uniform structured format.

Hence, the present disclosure (especially the methods and systems described herein) aims to provide mega-structuring all fragmented databases from different sources into datasets and unlock clinically relevant information, thereby enabling advanced analytics, clinical decision support, and/or precision medicine. Furthermore, structured datasets per disease may be obtained. For example, and similarly as describe above, the structured datasets per disease may be ultimately obtained in that the data engineering module together with the first machine learning model are used (especially after processing the at least one input data object by means of the data extraction and classification module).

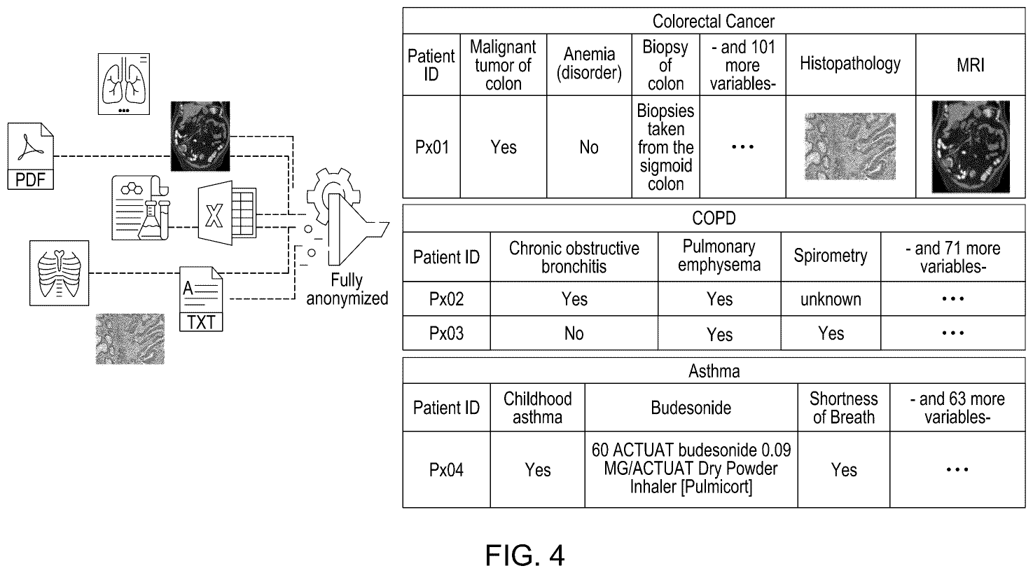

FIG. 4 shows an illustration of a data mega-structure according to an example. In the example, a clinically relevant database consists of a total ˜100,000 files, across 15 diseases, scattering in different types and formats (see lefthand side of FIG. 4). After processing, including filly anonymizing (see middle of FIG. 4), a structured datasets of variables per disease, which can be flexibly designed in any forms can be obtained (see righthand side of FIG. 4). There, examples are shown for colorectal cancer, COPD and asthma.

These preferred steps may improve diagnostic accuracy, reduces physician burnout, enhances precision treatment, and/or optimizes management across all levels of healthcare, from clinics and insurance companies to government.

The present disclosure (especially the methods and systems described herein) may also improve processing broad medical codes like “Encounter for problem”, for example, which may apply to multiple conditions, hence, making precise classification possible. Similarly, also names such as “Encounter Module Scheduled Wellness” which may be contained in multiple components that vary across documentation systems, may be processed in an improved manner, hence, leading to reduced inconsistencies. The present disclosure (especially the methods and systems described herein) aims to improve specificity and standardization, for example in that the data extraction and classification module is used. This allows, for example, that terms which have multiple meanings depending on the context, and variations in healthcare documentation can be understand in the correct way.

In an example, clinical data of cancer patients as an example can be stored in different hospitals'EHRs and originated from different sources. Important details, such as tumor diagnosis and progression, prior treatments, and doctors'notes, may be trapped in various file types and formats, some of which may be healthcare system notes, .pdf, handwritten notes, DICOM, and/or .xls files. Oncologists preferably need all data insights for a treatment plan but often spend a significant amount of time manually searching and reading, possibly in EHRs. This reality negatively impacts timely decision making, despite having EHRs. The present disclosure (especially the methods and systems described herein) aims to provide means for enabling the structure of a single consolidated data-table, or any another designed formats, which includes all meaningful data of not only one but all patients. For example, the data engineering module may be used to ultimately generate a uniform dataset containing the subject-related information in a uniform structured format. Hence, it is possible that oncologists can efficiently make the best fit treatment decision in a timely manner, instead of going through thousands of medical files with risk of missing important information.

The present disclosure (especially the methods and systems described herein) aims to enable fully clients/government-controlled healthcare data which may qualify diverse security and infrastructure needs, for example, by adhering to the strict data security, privacy, and regulatory standards, of each region/country.

Aspects (such as the methods and the computer systems described herein) according to the present disclosure (especially the methods and systems described herein) may be designed to automate the structure of medical data from multiple healthcare systems, handling various data types and formats. In particular, patient data may fragmented across multiple sources, including medical devices, departments, and even different hospitals. This fragmentation poses significant challenges for doctors in accessing and synthesizing all relevant information to make the best treatment decisions. Once integrated into the healthcare infrastructure, aspects according to the present disclosure (especially the methods and systems described herein) may allow to automatically collect these fragmented data, structuring them into a clinical mega-structured datasets.

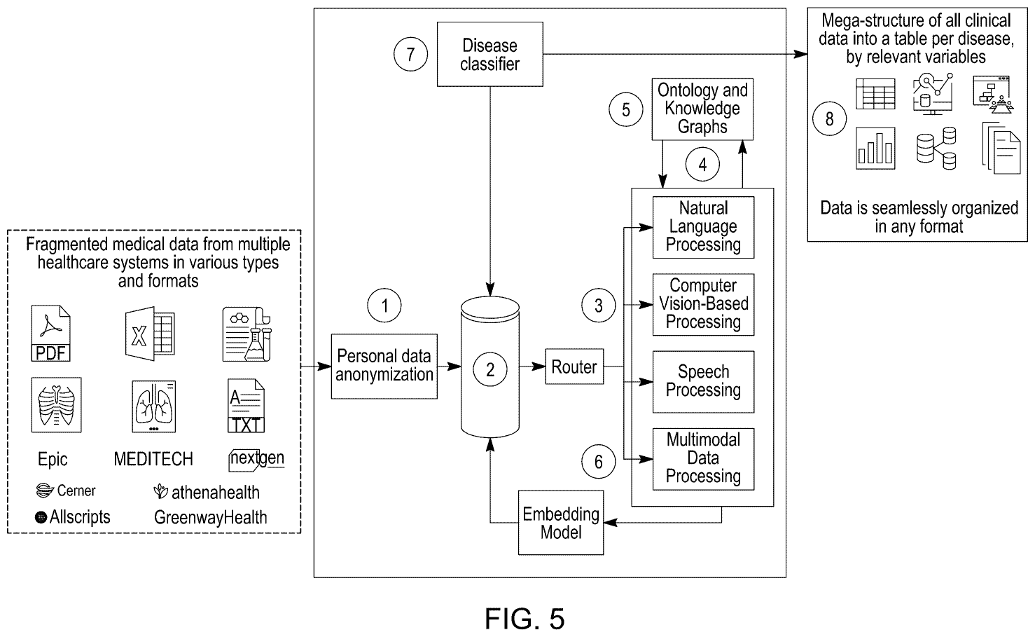

FIG. 5 shows, for example, an illustration of a workflow of a method according to an example. Every aspect denoted with numbers 1-8 may be used in preferred embodiments alone or in any combination. Hence, even though for the workflow of the exemplary method illustrated in FIG. 5 all aspects 1-8 are used together, in other embodiments only a selection of one or more aspects denoted with numbers 1-8 in FIG. 5 may be used.

In exemplary embodiments, the personal data anonymization module performs personal data anonymization (see for example No. 1 in the illustration of a workflow of a method according to an example in FIG. 5), thereby, for example, ensuring compliance with country/region data protection regulations, such as GDPR, HIPAA, and/or institutional policies. This process may deidentifies sensitive patient information, reducing the risk of unauthorized access or data breaches. In hospital settings, where data can be shared across departments and facilities, anonymization enables secure access to lab results, imaging scans, and treatment histories while maintaining confidentiality. For example, the present disclosure (especially the methods and systems described herein) allows seamless collaboration between radiology, cardiology, and oncology without exposing personal details. The personal data anonymization module may be configurable, allowing institutions to tailor privacy measures to their specific regulatory and operational needs.

For example, the personal data anonymization module may be applied prior to processing, by means of the data extraction and classification module of the computer system, the at least one input data object.

In exemplary embodiments, the anonymized data, such as the result of anonymization of personal data contained in the subject-related information, is stored in a high-performance object storage system (see for example No. 2 in the illustration of a workflow of a method according to an example in FIG. 5), which for example enables scalability, durability, and/or ability to manage diverse medical data types like imaging files, EHR records, and/or clinical notes. Object storage supports efficient indexing, metadata tagging, and integration with AI models and interoperability standards (FHIR, HL7). Depending on the institution's IT and security requirements, it can be deployed on-premises or in the cloud for greater scalability, accessibility, and full control.

In exemplary embodiments, especially following secure storage or as an alternative to secure storage, the system may intelligently route the data to specialized processing components based on its format and type (see for example No. 3 in the illustration of a workflow of a method according to an example in FIG. 5). This may comprise processing the at least one input data object (containing the subject-related information) by means of the data extraction and classification module, thereby classifying a data format of the input data object, performing a structure analysis on the input data object, extracting data from one or more detected components, detecting data extraction errors and correcting, combining extracted data from one or more components, and/or identifying domain specific entities and generating metadata. Some or all of these components may include natural language processing (NLP) for structured and unstructured text-based data, such as physician notes, discharge summaries, and/or pathology reports; computer vision for analyzing medical imaging, including X-rays, MRIs, and/or CT scans; speech processing for transcribing and interpreting audio-based clinical records, such as doctor-patient interactions and/or dictated reports; and/or multimodal processing for integrating complex, multi-source medical data streams (see for example No. 4 in the illustration of a workflow of a method according to an example in FIG. 5). For example, a radiology report containing both free-text descriptions and associated DICOM images can be processed (e.g. by means of the data extraction and classification module) using a combination of NLP and computer vision to extract clinical insights. Additionally, interoperability can be improved in exemplary embodiments by supporting standardized healthcare data formats like HL7, FHIR, and DICOM, enabling seamless integration across hospital information systems (HIS), electronic health records (EHRs), and picture archiving and communication systems (PACS). This structured routing allows that each data type may be processed in a preferred manner for downstream applications, such as clinical applications.

In exemplary embodiments, to enhance the accuracy and contextual relevance of structuring clinical variables, the data extraction and classification module and/or the data engineering module may incorporate ontology-based frameworks and/or knowledge graphs (see for example No. 5 in the illustration of a workflow of a method according to an example in FIG. 5). This way the understanding of medical terminologies, relationships between clinical entities, and/or disease-specific variations contained in the subject-related information may be enriched. By leveraging standardized ontologies like LOINC, SNOMED-CT, RxNorm, and ICD-10, the present disclosure (especially the methods and systems described herein) may improve interoperability across EHRs and clinical databases and/or data consistency. This approach may mitigate AI hallucinations, for example by constraining outputs within validated medical knowledge, reducing errors and misinterpretations. For example, in oncology, the present approach may differentiate between similar terms like ‘neoplasm’ and ‘benign lesion,’, hence improving preciseness clinical insights. Additionally, said knowledge graphs may improve inferential reasoning, helping identify related conditions, drug interactions, and/or disease progression patterns. This may improve clinical accuracy, supports decision-making, and ensures standardized data representation across diverse healthcare environments.

In exemplary embodiments, the subject-related information may be further refined and transformed through an advanced embedding model (see for example No. 6 in the illustration of a workflow of a method according to an example in FIG. 5), which may be part of the data extraction and classification module. The advanced embedding model may, for example, convert complex medical information into structured representations optimized for downstream analytics and/or predictive modeling. To this end, in an embodiment, the advanced embedding model is employed at least for the structure analysis on the input data object. The described process may improve pattern recognition, enabling more accurate disease classification, risk stratification, and/or treatment response predictions. The refined data (for example, after subsequent generating the uniform dataset by means of the data engineering module), may then systematically stored in a structured format, especially by means of the storing module. This way efficient retrieval for clinical interpretation, decision support, and/or integration with AI-driven applications, such as automated diagnostics and/or personalized treatment recommendations may be improved.

In exemplary embodiments, disease-relevant clinical variables may be extracted (see for example No. 7 in the illustration of a workflow of a method according to an example in FIG. 5) and structured into comprehensive, condition-specific datasets (see for example No. 8 in the illustration of a workflow of a method according to an example in FIG. 5), especially by means of the data extraction and classification module. This way standardized and interpretable data for clinical use may be improved. These curated datasets may enable precise and efficient assessments by ultimately providing a consolidated view of patient health, supporting differential diagnosis, treatment planning, and/or outcome prediction. Additionally, they may facilitate large-scale medical research, improve predictive analytics by enhancing AI model training with high-quality inputs, and/or integrate seamlessly with decision support systems for real-time clinical guidance.

By transforming fragmented data into structured, actionable insights, the present disclosure (especially the methods and systems described herein) may improve healthcare intelligence, optimizes operational workflows, and/or may strengthens evidence-based medical decision-making across diverse healthcare settings.

The capability of the present disclosure (especially the methods and systems described herein) to structure unstructured medical fragmented data from various types and formats into datasets, may incorporate using a Large Language Model to enrich the clinical context. The enriched data may then be transformed into multiple widely used healthcare formats, including FHIR, HL7, CSV, PDF, TXT, as well as unstructured formats such as free-text clinical notes and imaging reports.

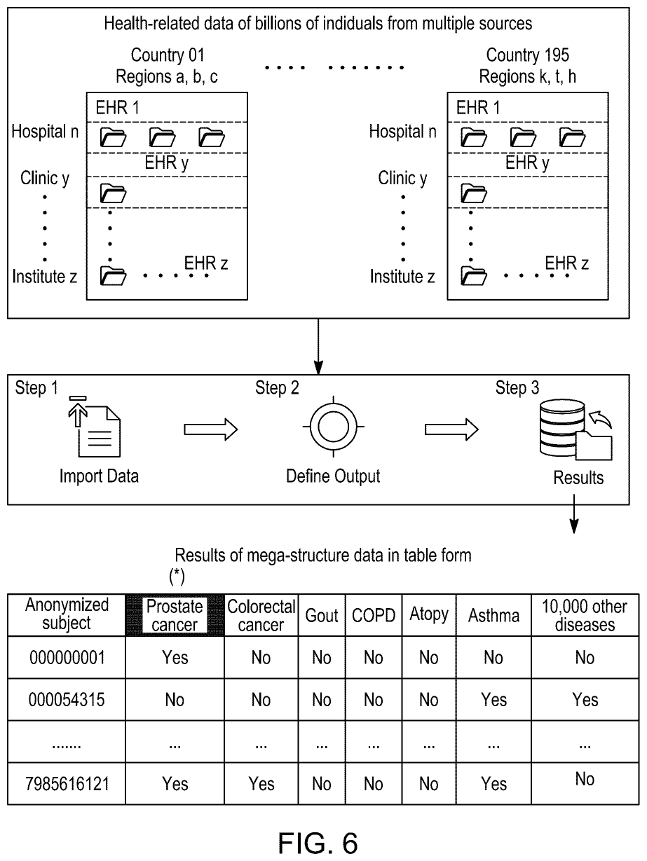

FIG. 6-9, which will be described in more detail below, show examples of data mega-structure (even from billions of individuals).

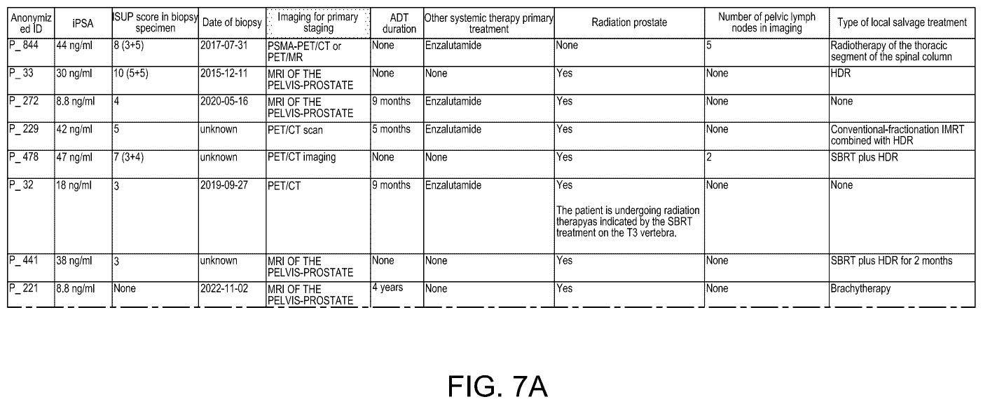

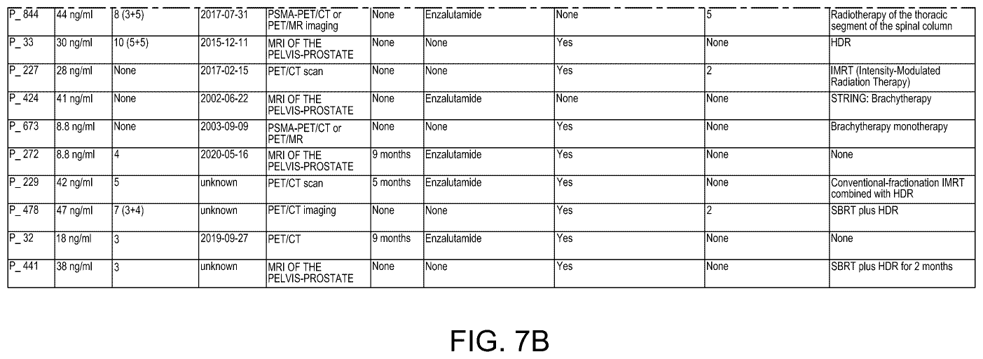

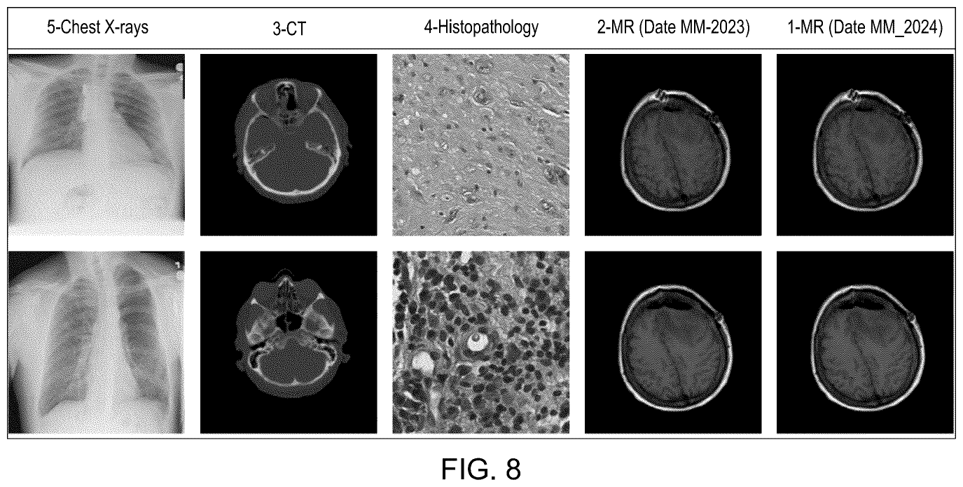

FIG. 6 shows an illustration of a data mega-structure according to another example. In an exemplary embodiment, the present disclosure (especially the methods and systems described herein) aims to provide mega-structure health-related data of billions of individuals from multiple sources, such as hospitals, clinics, and/or institutions, in different regions and countries, for example as illustrated in FIG. 6. For example, all kinds of data may be processed (for example in the method and by the computer system of the present disclosure), which for, example, may comprise at least one of image information, textual information, acoustic information, voice information, spreadsheet data, and/or database information. The database information may include at least one of data generated by software and/or hardware, relational database information, object-oriented database information, and/or NoSQL database information. The data may also be doctors notes, blood tests, clinical images, such as MRI, PET, drug treatments, and/or other tests. The data may, alternatively or in addition, be structured and/or unstructured data organized and/or stored in any applications, such as electronic health records, and/or protected by data security platforms. For example, the in the context of the present disclosure, processing and mega-structuring data in a table format, or other formats, such as knowledge graph, chart, and others may be provided. In exemplary embodiments, the process and mega-structure can be as simple as three steps (see for example FIG. 6), including a first step: importing data (for example: the data may be received in the method and/or by the computer system of the present disclosure); a second step: defining output (for example, a selection of mega-structure format, such as table format may be part of the method and/or of the computer system of the present disclosure); and a third step: Obtaining results of the data mega-structure. The results of mega-structure data in table form may structure data according to anonymized identification numbers and classification of diseases. The results may be mega-structured according to other information which is designed by users. The total anonymized identification numbers may be eight billions of individuals or more, and the total number of diseases may be 10,000 or more. A table of each disease may be shown when selecting the name of such disease (for example in the method and/or in the computer system of the present disclosure). For example, a selection of prostate cancer (marked by “(*)” in FIG. 6) may open a table which mega-structures all meaningful clinical variables, as shown in exemplary way in FIG. 7. The clinical variables may be designed by the users, and other information may also be included for mega-structure. Furthermore, a selection of imaging for primary staging may open a table which mega-structures all clinical imaging data, such as MRI, histopathology, being presented in the same page, as shown in exemplary way in FIG. 8. The present disclosure, in an embodiment, may provide clinical image evaluation methods. Furthermore, the present disclosure, in an embodiment, may provide presenting all raw and/or original data which was mega-structured.

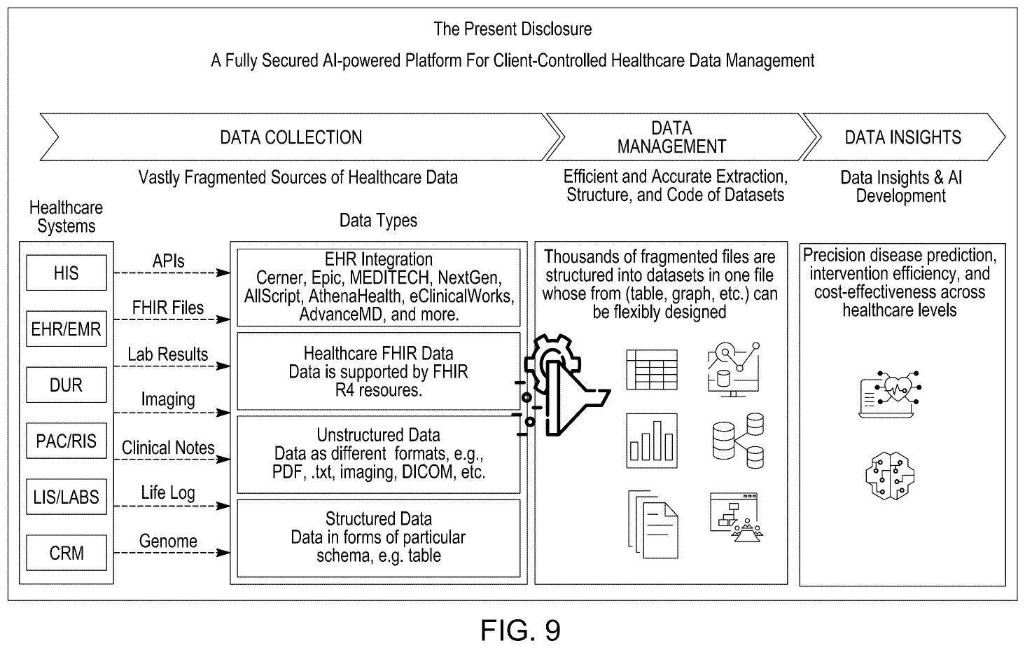

FIG. 9 shows an illustration of a fully secured AI-powered platform according to an example. For example, the platform may be realized as method or system according to the present disclosure. In that respect, in exemplary embodiments of the present disclosure (for example of the methods and systems as described herein) mega-structuring a largely fragmented clinical database into a comprehensive table of all anonymized patients, and variables per disease is possible. The clinical data mega-structure may be designed in any forms beyond table, such as charts, graphs, knowledge interaction, and more. This further provides an improved accuracy for clinical data semantic search and management across various sources. The present disclosure (especially the methods and systems described herein) aims supporting both on-premises and cloud-based servers. This may allow full clients/government-controlled healthcare data, and qualifies diverse security and infrastructure needs by adhering to the strict data security, privacy, and/or regulatory standards, such as such as HIPAA and GDPR, of each region/country.

The above examples have been described mainly in a health-related context. However, it will be understood that some, or all, of the above-described functionalities and advantages are achievable also in connection with other subject-related fields of data development.

What is claimed is:

1. A method, performed by a computer system connected to a network, the method comprising:
receiving, by means of a data receiving module of the computer system and from at least one of a plurality of sources connected to the network, at least
one input data object containing subject-related information according to at least one of a plurality of information types encoded in at least one of a plurality of data formats;
processing, by means of a data extraction and classification module of the computer system, the at least one input data object for standardizing the subject-related information;
subjecting, by means of a data engineering module of the computer system, the subject-related information contained in the processed at least one input data object to a first machine learning model for generating a uniform dataset containing the subject-related information in a uniform structured format;
storing, by means of a storing module of the computer system, the uniform dataset in one or more secured data repositories connected to the network; and
providing, by means of a workspace module of the computer system, a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.

2. The method according to claim 1, wherein the plurality of information types comprises at least one of image information, textual information, acoustic information, voice information, spreadsheet data, and/or database information, the database information including at least one of data generated by software and/or hardware, relational database information, object-oriented database information, and/or NoSQL database information.

3. The method according to claim 1, wherein the uniform dataset is generated such that the uniform dataset complies with at least one privacy and/or security standard defined by legal regulations in one or more jurisdictions regarding the subject-related information.

4. The method according to claim 1, wherein the method comprises: Mega-structuring data, such as the subject-related information contained in the at least one input data object, from multiple sources, especially of any kinds, and/or from any management levels, such as clinics, hospitals, ministry of health, and/or countries, into a structured formats, such as a table form.

5. The method according to claim 4, wherein the mega-structuring data provide for one or more of the following information and/or functionalities a.-f., for example, at least in part in form of a spreadsheet table:
a. A column, such as a first column, is an anonymized subjects'number from 1 to N, with for example N=361,742,591,
b. A horizontal row imports all defined diseases, indicating how many diseases a person suffers from and/or how the disease spreads within the population, wherein especially a summary for each disease is provided at the bottom of all subjects,
c. Pressing on a disease X (cancer, for example) opens a spread that indicates data and/or health-related data, such as symptoms, blood tests, MRIs, PET, drug treatments, imaging, PDFs, and/or other tests, wherein especially a column, such as the first column, may be the anonymous subject number of the tests and the rows,
d. Pressing on an information, such as an MRI image, opens the history of all information related to that information, such as further MRI images, for each subject, and/or wherein pressing the drug treatment presents all the concomitant drugs the subject takes,
e. In a separate table, diagnosis, prognosis, drug side effects, morbidity, etc. are provided, and/or
f. Suitability of the result data, such as a structured table of health-related data, to be used in large-scale medical research, and/or to be used during training of AI model, especially for improving predictive analytics by enhancing AI model training.

6. The method according to claim 1, wherein the subject-related information contained in the received at least one input data object is subjected to a personal data anonymization module, for performing anonymization of personal data contained in the subject-related information, especially prior to processing, by means of the data extraction and classification module of the computer system, the at least one input data object.

7. The method according to claim 1, wherein standardizing the subject-related information comprises applying an error-detection-and-correction routine to the subject-related information.

8. The method according to claim 1, wherein the plurality of sources comprises a plurality of source types.

9. The method according to claim 1, wherein the uniform structured format comprises a uniform category-mapped format, in particular a uniform category-mapped tabular, diagram, chart and/or figure format.

10. The method according to claim 1, wherein the data development operations include subjecting the imported datasets to at least one second machine learning model comprised by the workspace module.

11. The method according to claim 1, wherein:
the subject-related information is life sciences-related, in particular health-related, information, and
the subject-related data development operations are life sciences-related, in particular health-related, data development operations.

12. The method according to claim 1, wherein:
the subject-related information is information from multiple domains, in particular comprising life sciences-related, more particularly health-related, health maintenance organizations-related, pharmaceutical technologies-related, biology-related and/or bio-technologies-related information, and
the subject-related data development operations are for data from multiple domains and comprise life sciences-related, in particular health-related, health maintenance organizations-related, pharmaceutical technologies-related, biology-related and/or bio-technologies-related data development operations.

13. The method according to claim 11, wherein the user-controlled subject-related data development operations comprise receiving, from a user via the network, a life sciences-related, in particular health-related, pharmaceutical technologies-related, biology-related, biotechnologies-related, query, and the at least one workspace-developed data object comprises a life sciences-related, in particular health-related, pharmaceutical technologies-related, biology-related, biotechnologies-related, response to the life sciences-related query, the life sciences-related response for output by the computer system.

14. The method according to claim 13, wherein the life sciences-related query is a health-related query and the life sciences-related response is a health-related response, the health-related query and the health-related response relating to at least one of a clinical condition and information of a person, medical information, a description of a medication, an interaction of medications, a mechanism of action of a medication, an underlying cause of a disease, a prediction of a disease development, a prevention of a disease development, medical treatment, personalized treatment, best fit treatment, a biological target for a treatment, and/or drug development.

15. The method according to claim 11, wherein the plurality of information types comprises at least one of a handwritten note by a medical professional, a medical image, an electronic healthcare record, medical spreadsheet data, and/or medical database information, the medical database information including at least one of clinical data generated by software and/or hardware, relational database information, object-oriented database information, and/or NoSQL database information.

16. The method according to claim 1, wherein the one or more secured data repositories comprise one or more secured data repositories hosted by an operator of the computer system and/or one or more secured data repositories hosted at one or more secured user domains of one or more users of the computer system.

17. A computer program product containing portions of program code which, when executed by a processor of a computer system, configure the computer system to perform the method of claim 1.

18. A computer system comprising a processor and a data storage device operatively coupled to the processor, the data storage device containing portions of program code which, when executed by the processor, configure the processor to perform the following steps:
receiving, by means of a data receiving module of the computer system and from at least one of a plurality of sources connected to a network, at least one input data object containing subject-related information according to at least one of a plurality of information types encoded in at least one of a plurality of data formats;
processing, by means of a data extraction and classification module of the computer system, the at least one input data object for standardizing the subject-related information;
subjecting, by means of a data engineering module of the computer system, the subject-related information contained in the processed at least one input data object to a first machine learning model for generating a uniform dataset containing the subject-related information in a uniform structured format;
storing, by means of a storing module of the computer system, the uniform dataset in one or more secured data repositories connected to the network; and
providing, by means of a workspace module of the computer system, a secured virtual environment accessible to users connected to the network, the secured virtual environment enabling importation of datasets stored in the one or more secured data repositories and a use of imported datasets as part of one or more user-controlled subject-related data development operations for generating at least one workspace-developed data object.
